(19) United States
(12) Patent Application Publication
(10) Pub. No.: US 2025/0235204 A1
(43) Pub. Date: Jul. 24, 2025

(54) SUTURE LOCKING SYSTEMS FOR PERFORMING TENSIONABLE KNOTLESS SURGICAL PROCEDURES

(71) Applicant: ARTHREX, INC., Naples, FL (US)

(72) Inventors: Andrew Christian PETRY, Naples, FL (US); Peter J. DREYFUSS, Naples, FL (US); Patrick Joel DENARD, Jacksonville, OR (US)

(21) Appl. No.: 18/421,257

(22) Filed: Jan. 24, 2024

Related U.S. Application Data
(60) Provisional application No. 63/622,978, filed on Jan. 19, 2024.

(51) Int. Cl. A61B 17/04; A61B 17/88

(52) U.S. Cl. CPC A61B 17/0487; A61B 17/0401; A61B 17/8869; A61B 2017/0404

(57) ABSTRACT

Systems and methods are provided for performing tensionable knotless surgical procedures. A locking ferrule that includes a one-way locking mechanism may be utilized for tensioning and locking one or more strands of suture during the surgical procedure. The one-way locking mechanism may be established by one or more locking barbs of the suture locking device.

### CROSS-REFERENCE TO RELATED APPLICATIONS

This application claims the benefit of U.S. Provisional Application No. 63/622,978, which was filed on Jan. 19, 2024 and is incorporated hereby by reference in its entirety.

### BACKGROUND

This disclosure relates to the field of surgery, and more particularly to various suture locking systems that are capable of locking suture when performing a variety of tensionable knotless surgical repairs.

Repetitive trauma to a joint, such as a knee, ankle, hip, elbow, or shoulder joint, for example, may result in the development of tissue defects (e.g., soft tissue tears, cartilage defects, etc.). If not treated, tissue defects could further deteriorate, thereby causing joint instability and discomfort.

### SUMMARY

This disclosure relates to systems and methods for performing tensionable knotless surgical procedures, such as tissue repairs, for example. Locking ferrules that include a one-way locking mechanism may be utilized when performing the tensionable knotless surgical procedures for tensioning and locking one or more strands of suture.

An exemplary suture locking system may include, inter alia, a surgical button, a locking ferrule that includes a one-way locking mechanism, a flexible coupling that extends between the surgical button and the locking ferrule, and a suture received through the locking ferrule and locked relative to the locking ferrule by the one-way locking mechanism.

Another exemplary suture locking system may include, inter alia, a bone plate including an opening, a locking ferrule received within the opening and including a one-way locking mechanism, and a suture received through the locking ferrule and locked relative to the bone plate by the one-way locking mechanism.

Yet another exemplary suture locking system may include, inter alia, an arthroplasty implant, a locking ferrule connected to the arthroplasty implant and including a one-way locking mechanism, and a shuttle device passed through the locking ferrule.

The embodiments, examples, and alternatives of the preceding paragraphs, the claims, or the following description and drawings, including any of their various aspects or respective individual features, may be taken independently or in any combination. Features described in connection with one embodiment are applicable to all embodiments, unless such features are incompatible.

The various features and advantages of this disclosure will become apparent to those skilled in the art from the following detailed description. The drawings that accompany the detailed description can be briefly described as follows.

### BRIEF DESCRIPTION OF THE DRAWINGS

FIG. 1 illustrates an exemplary suture locking system.

FIG. 2 is a cross-sectional view through section 2-2 of FIG. 1.

FIG. 3 illustrates a suture having a varying thickness.

FIGS. 4, 5, and 6 schematically illustrate a surgical method for performing a tissue repair using the suture locking system of FIGS. 1 and 2.

FIG. 7 illustrates another exemplary suture locking system.

FIG. 8 schematically illustrate a surgical method for performing a tissue repair using the suture locking system of FIG. 7.

FIG. 9 illustrates an exemplary locking ferrule.

FIG. 10 illustrates another exemplary locking ferrule.

FIG. 11 illustrates yet another exemplary suture locking system.

FIGS. 12, 13, and 14 illustrates a surgical method for performing a tissue repair using the suture locking system of FIG. 11.

### DETAILED DESCRIPTION

This disclosure relates to systems and methods for performing tensionable knotless surgical procedures. A locking ferrule that includes a one-way locking mechanism may be utilized as part of the tensionable knotless repairs for tensioning and locking one or more strands of suture. These and other features of this disclosure are described in further detail below.

An exemplary suture locking system may include, inter alia, a surgical button, a locking ferrule that includes a one-way locking mechanism, a flexible coupling that extends between the surgical button and the locking ferrule, and a suture received through the locking ferrule and locked relative to the locking ferrule by the one-way locking mechanism.

In any further embodiment, the one-way locking mechanism is configured to allow the suture to be tensioned in a first direction and prevent the suture from moving in a second direction.

In any further embodiment, the one-way locking mechanism is established by a plurality of locking barbs provided within a cannulation of the locking ferrule.

In any further embodiment, the flexible coupling is adjustable to reduce a distance between the surgical button and the locking ferrule.

In any further embodiment, the flexible coupling is a second suture that is separate and distinct from the suture.

In any further embodiment, the flexible coupling is an adjustable suture loop.

Another exemplary suture locking system may include, inter alia, a bone plate including an opening, a locking ferrule received within the opening and including a one-way locking mechanism, and a suture received through the locking ferrule and locked relative to the bone plate by the one-way locking mechanism.

In any further embodiment, the one-way locking mechanism is configured to allow the suture to be tensioned in a first direction and prevent the suture from moving in a second direction.

In any further embodiment, the one-way locking mechanism is established by a plurality of locking barbs provided within a cannulation of the locking ferrule.

In any further embodiment, the cannulation extends through an eyelet portion of the locking ferrule.

In any further embodiment, an engagement portion is connected to the eyelet portion.

In any further embodiment, the engagement portion includes a first thread that is configured to engage a second thread of the opening of the bone plate.

In any further embodiment, the locking ferrule includes an engagement portion received within the opening and an eyelet portion that extends to a location outside of the opening.

In any further embodiment, the eyelet portion provides the one-way locking mechanism.

In any further embodiment, the one-way locking mechanism includes a plurality of locking barbs that allow the suture to be tensioned in a first direction and prevent the suture from moving in a second direction.

Yet another exemplary suture locking system may include, inter alia, an arthroplasty implant, a locking ferrule connected to the arthroplasty implant and including a one-way locking mechanism, and a shuttle device passed through the locking ferrule.

In any further embodiment, the shuttle device is configured to shuttle a suture through the locking ferrule.

In any further embodiment, the shuttle device includes an eyelet and a free end.

In any further embodiment, the one-way locking mechanism is configured to allow the suture to be tensioned in a first direction and prevent the suture from moving in a second direction.

In any further embodiment, the one-way locking mechanism is established by a plurality of locking barbs provided within a cannulation of the locking ferrule.

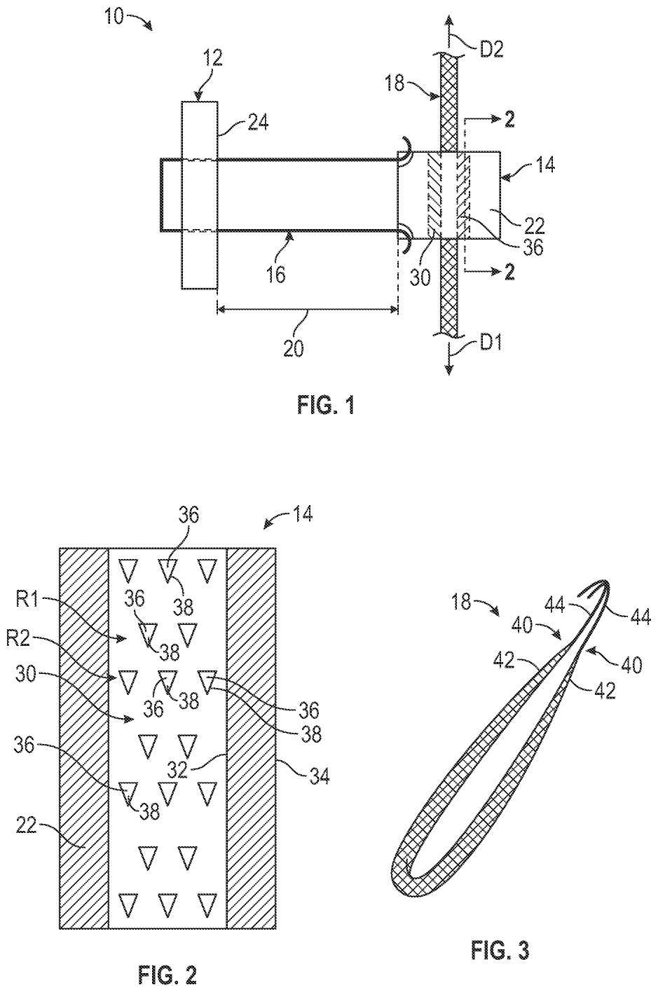

FIGS. 1-2 illustrate an exemplary suture locking system 10 that can be used when performing various tensionable knotless surgical procedures. For example, the suture locking system 10 could be utilized during surgical methods for performing tissue repairs. The suture locking system 10 could be used in conjunction with a variety of orthopedic surgical repairs, including but not limited to cerclage procedures, subscapularis repairs, syndesmosis repairs, fracture repairs, AC Joint repairs, among many others.

The suture locking system 10 may include a surgical button 12, a locking ferrule 14, a flexible coupling 16 that extends between the surgical button 12 and the locking ferrule 14, and a suture 18. In an embodiment, the surgical button 12 is an elongated or oblong-shaped button. However, the size, shape, and material-makeup of the surgical button 12 are not intended to limit this disclosure.

The flexible coupling 16 may be connected to both the surgical button 12 and the locking ferrule 14 in any known manner. For example, the surgical button 12 and the locking ferrule 14 may each include one or more openings for accommodating portions of the flexible coupling 16. The flexible coupling 16 may be tensioned or otherwise adjusted to alter a distance 20 between the surgical button 12 and the locking ferrule 14. For example, the flexible coupling 16 may be adjusted to reduce the distance 20 between the surgical button 12 and the locking ferule 14.

In an embodiment, the flexible coupling 16 is another suture that is a separate and distinct suture strand from the suture 18. In another embodiment, the flexible coupling 16 is an adjustable suture loop. Other configurations are contemplated within the scope of this disclosure.

The locking ferrule 14 may include a tubular body 22. A cannulation 30 may extend through the tubular body 22 for establishing a through-passage for accommodating one or more strands of the suture 18. The cannulation 30 may be circumscribed by an inner diameter wall 32 of the tubular body 22. The cannulation 30 may be straight or may taper toward one direction. An outer diameter wall 34 of the tubular body 22 may provide a smooth surface.

A plurality of locking barbs 36 may protrude inwardly from the inner diameter wall 32 of the tubular body 22. The locking barbs 36 may therefore occupy at least a portion of the open space of the cannulation 30. In an embodiment, the locking barbs 36 are integrally formed (e.g., molded) features of the tubular body 22. The locking barbs 36 may be provided along an entire length of the cannulation 30 or at only select portions thereof. The locking barbs 36 may be either rigid or flexible structures.

The locking barbs 36 may be arranged in multiple rows along the length of the cannulation 30. For example, the locking barbs 36 may be arranged in a least a first row R1 and a second row R2 (see FIG. 2). In an embodiment, the locking barbs 36 of the second row R2 are staggered relative to the locking barbs 36 of the first row R1 (see FIG. 2).

Each locking barb 36 may include a sharp or pointed tip 38, and each locking barb 36 may be angled in the same direction, such as toward a common end of the tubular body 22, for example. The locking barbs 36 may therefore establish a one-way locking mechanism that permits the suture 18 to pass through the cannulation 30 in a first direction D1 while preventing the suture 18 from being tensioned or otherwise moved in a second direction D2. The staggered relationship of the adjacent rows of locking barbs 36 may provide for maximum engagement with the suture 18 once it has been passed through the cannulation 30.

The suture 18 may be FiberWire®, FiberTape®, or any other suitable suture product. FiberWire® and FiberTape® are suture products marketed and sold by Arthrex, Inc. However, other suture products could be utilized within the scope of this disclosure. The size and type of suture utilized in conjunction with the locking ferrule 14 of the suture locking system 10 are not intended to limit this disclosure.

The suture 18 may include a varying thickness. The suture 18 may therefore include one or more tapered regions 40 where the suture 18 transitions between a thickened section 42 and a thinned section 44 (see FIG. 3). The thinned sections 44 can facilitate passing the suture 18 through the cannulation 30, such as via a suitable suture loader that can be provided as part of the suture locking system 10, for example, and the thickened sections 42 can provide greater surface area for the locking barbs 36 to engage in order to sufficiently lock the suture 18 to the locking ferrule 14 and thus relative to the surgical button 12.

In an embodiment, the thickened sections 42 of the suture 18 are about twice as thick as the thinned sections 44. However, other ratios (e.g., 1.5:1, 3:1, etc.) between the relative thicknesses (e.g., outer diameters) of the thickened section 42 and the thinned section 44 are contemplated within the scope of this disclosure.

Notably, in the above implementations, the cannulation 30 extends along an axis that is substantially parallel to a top surface 24 of the surgical button 12. Accordingly, the suture 18 can be tensioned in a direction that is parallel with the top surface 24 of the surgical button 12. However, the cannulation 30 could alternatively be configured such that the suture 18 is passed and tensioned along a direction that is transverse (e.g., substantially perpendicular) to the top surface 24 of the surgical button 12.

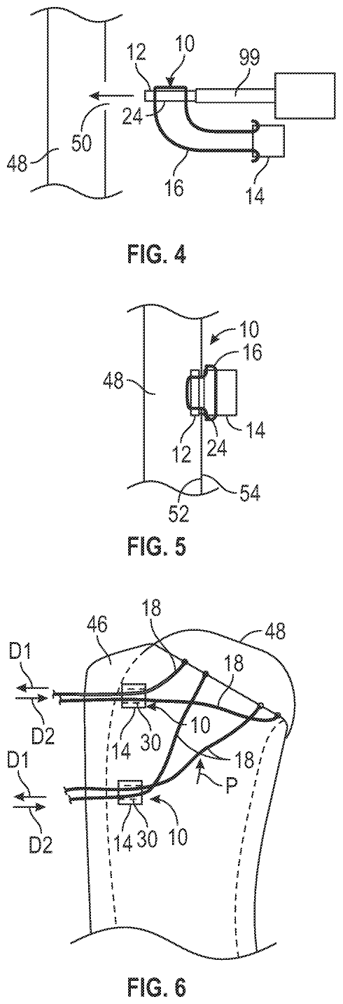

FIGS. 4, 5, and 6 schematically illustrate an exemplary surgical method in which the suture locking system 10 of FIGS. 1-2 may be utilized to tension and lock the suture 18 during a surgical repair (e.g., soft tissue-to-bone fixation, etc.). In this embodiment, the surgical method may involve securing a tissue 46 (e.g., a subscapularis muscle) to a bone 48 (e.g., a humerus).

Referring first to FIG. 4, the surgical method may include preparing a unicortical socket 50 at a desired location of the bone 48. The surgical button 12 of the suture locking system 10 may be inserted into the bone 48 (e.g., by an inserter device 99) through the unicortical socket 50. The surgical button 12 may be inserted into the unicortical socket 50 in a first configuration (see FIG. 4) and may then be flipped to a second, transverse configuration (see FIG. 5) for achieving fixation relative to the bone 48 once the surgical button 12 has cleared the inner end of the unicortical socket 50. When fixated relative to the bone 48, the top surface 24 of the surgical button 12 is received against an interior surface 52 of the bone 48.

Referring to FIG. 5, the flexible coupling 16 may be tensioned or otherwise adjusted to reduce the distance 20 between the surgical button 12 and the locking ferrule 14. In the reduced position, the locking ferrule 14 may be received against or in close proximity to an exterior surface 54 of the bone 48. In some embodiments, the locking ferrule 14 could be received directly over top of the tissue 46 that is to be fixated to the bone 48 in the reduced position.

Referring to FIG. 6, one or more sutures 18 may be passed through, around or otherwise secured relative to the 46 and may then be passed through the cannulation 30 of the locking ferrule 14. The sutures 18 may then be tensioned in the first direction D1 to knotlessly lock the suture 18 relative to the locking ferrule 14 and thereby secure the tissue 46 at a desired position relative to the bone 48. Tensioning the sutures 18 in the direction D1 allows the pointed tips 38 of the locking barbs 36 to interdigitate with the sections of the sutures 18 that are accommodated within the cannulation 30, thereby locking the sutures 18 in place and preventing them from sliding back in the second direction D2.

In some implementations, a plurality of the suture locking systems 10 may be implanted into the bone 48 as part of the surgical method for securing the tissue 46 to the bone 48. The sutures 18 may be arranged in various bridging configurations over top of the tissue 46 by the locking ferrules 14. For example, as shown in FIG. 6, the sutures 18 may be arranged in a crisscross pattern P that provides a desired area of footprint compression over top of the tissue 46. Any bridging configuration using any number of suture locking systems 10 and sutures 18 can be achieved as part of the surgical method.

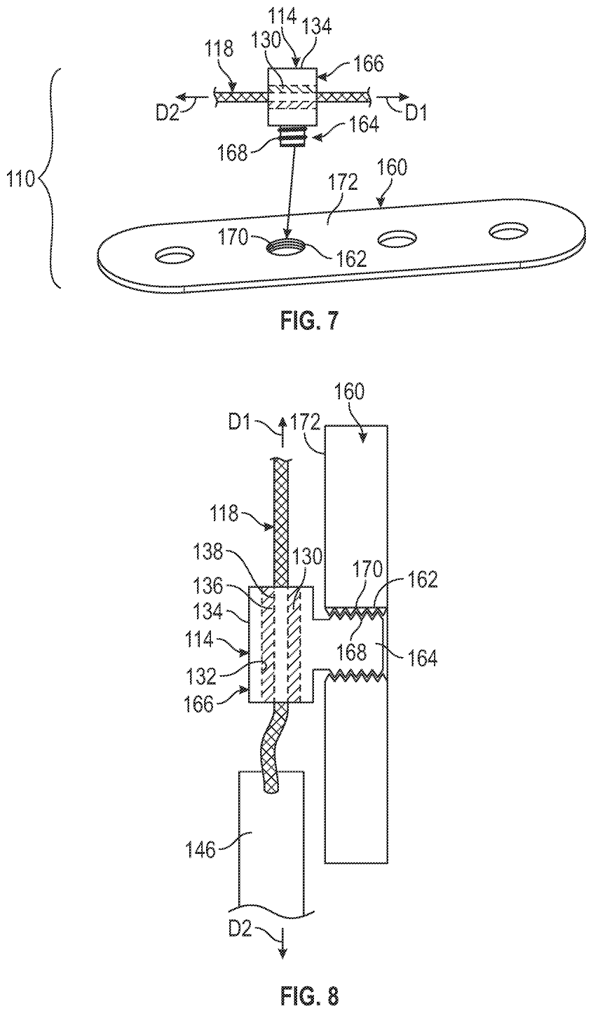

FIGS. 7 and 8 illustrate another exemplary suture locking system 110 that can be used when performing various tensionable knotless surgical procedures. The suture locking system 110 may include a bone plate 160, one or more locking ferrules 114 that are removably securable relative to the bone plate 160, and one or more sutures 118. The size, shape, and material-makeup of the bone plate 160 are not intended to limit this disclosure.

The bone plate 160 may include one or more openings 162 formed through a plate body of the bone plate 160. The openings 162 may establish through-passages for accommodating other devices, such as the locking ferrule 114 and/or screws.

The locking ferrule 114 of the suture locking system 110 may include an engagement portion 164 and an eyelet portion 166. The engagement portion 164 may be configured to connect to one of the openings 162, such as via a threaded engagement. For example, the engagement portion 164 may include a first thread 168 that is configured to engage a second thread 170 of the opening 162 for securing the locking ferrule 114 to the bone plate 160. When the engagement portion 164 is secured to the bone plate 160, the eyelet portion 166 extends outside of the opening 162 (e.g., slightly above or proud of a top surface 172 of the bone plate 160) and may be configured for receiving and locking the suture 118 relative to the bone plate 160.

A cannulation 130 may extend through the eyelet portion 166 of the locking ferrule 114. The cannulation 130 may establish a through-passageway for accommodating one or more strands of the suture 118. The cannulation 130 may be circumscribed by an inner diameter wall 132 of the eyelet portion 166. An outer diameter wall 134 of the eyelet portion 166 may provide a substantially smooth surface.

A plurality of locking barbs 136 may protrude inwardly from the inner diameter wall 132 of the eyelet portion 166. The locking barbs 136 may therefore occupy at least a portion of the open space of the cannulation 130. The locking barbs 136 may be arranged in one or more rows along the length of the cannulation 130. Each locking barb 136 may include a sharp or pointed tip 138, and each locking barb 136 may be angled in the same direction, such as toward a common end of the eyelet portion 166. The locking barbs 136 may therefore establish a one-way locking mechanism that permits the suture 118 to pass through the cannulation 30 in a first direction D1 while preventing the suture 118 from being tensioned or otherwise moved in a second direction D2.

Either before or after securing the locking ferrule 114 in place within one of the openings 162 of the bone plate 160, the suture 118 may be passed through, around, or otherwise secured relative to a tissue 146 (e.g., bone, ligament, tendon, muscle, etc., see FIG. 8) and then be passed through the eyelet portion 166 of the locking ferrule 114. The suture 118 may subsequently be tensioned to knotlessly secure the tissue 146 at a desired position relative to the bone plate 160. Tensioning the suture 118 in the first direction D1 allows the pointed tips 138 of the locking barbs 136 to interdigitate with the portion of the suture 118 accommodated within the cannulation 130, thereby locking the suture 118 in place and preventing it from sliding back in the second direction D2.

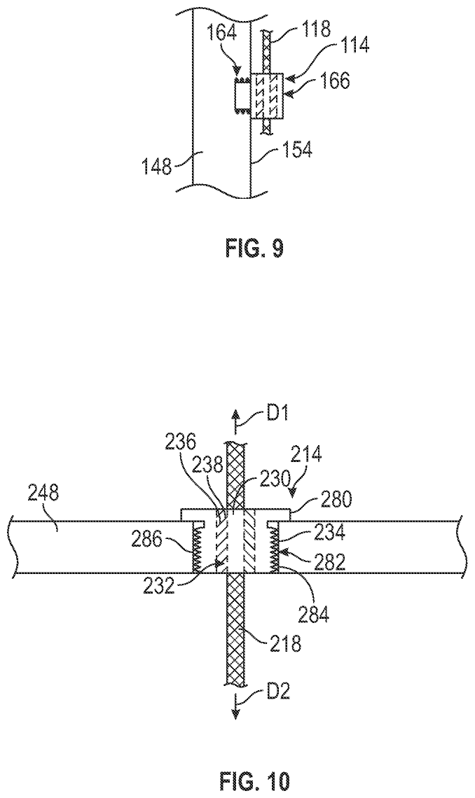

The locking ferrule 114 described above could alternatively be utilized independently from the bone plate 160. For example, as shown in FIG. 9, the engagement portion 164 of the locking ferrule 114 may be secured (e.g., screwed) directly into a bone 148. When the engagement portion 164 is secured to the bone 148, the eyelet portion 166 extends slightly above or proud of an exterior surface 154 of the bone 148 and may be configured for receiving and locking the suture 118 relative to the bone 148.

Another exemplary locking ferrule 214 that could be utilized independently from either a bone plate or a surgical button is illustrated in FIG. 10. The locking ferrule 214 may include a cap portion 280 and a barrel portion 282 that protrudes from the cap portion 280. The cap portion 280 and the barrel portion 282 may be formed together as part of a unitary structure of the locking ferrule 214. In an embodiment, the cap portion 280 and the barrel portion 282 are arranged to provide a top hat-like appearance of the locking ferrule 214. However, the specific size, shape, and material make-up of the locking ferrule 214 are not intended to limit this disclosure.

The barrel portion 282 may include an inner diameter wall 232 and an outer diameter wall 234. The outer diameter wall 234 may include a bone engagement feature 286 (e.g., threads, barbs, ledges, etc.) for facilitating bone fixation. For example, the locking ferrule 214 may be positioned relative to a bone 248 such that the barrel portion 282 is accommodated within a socket or tunnel 284 formed in a bone 248 and the cap portion 280 is seated relative to the cortex of the bone 248.

A cannulation 230 may extend through the cap portion 280 and the barrel portion 282. The cannulation 230 may establish an internal passageway for accommodating one or more strands of suture 218. The cannulation 230 may be circumscribed by the inner diameter wall 232. A plurality of locking barbs 236 may protrude inwardly from the inner diameter wall 232. The locking barbs 36 may therefore occupy at least a portion of the open space of the cannulation 230. Each locking barb 236 may include a sharp or pointed tip 238, and, in this implementation, each locking barb 236 may be angled in a direction toward the cap portion 280. The locking barbs 236 may therefore establish a one-way locking mechanism that permits the suture 218 to pass through the cannulation 230 in a first direction D1 while preventing the suture 218 from being tensioned or otherwise moved in a second direction D2. The suture 218 may be tensioned to move the bone 248 into a desired position, such as relative to another bone, for example.

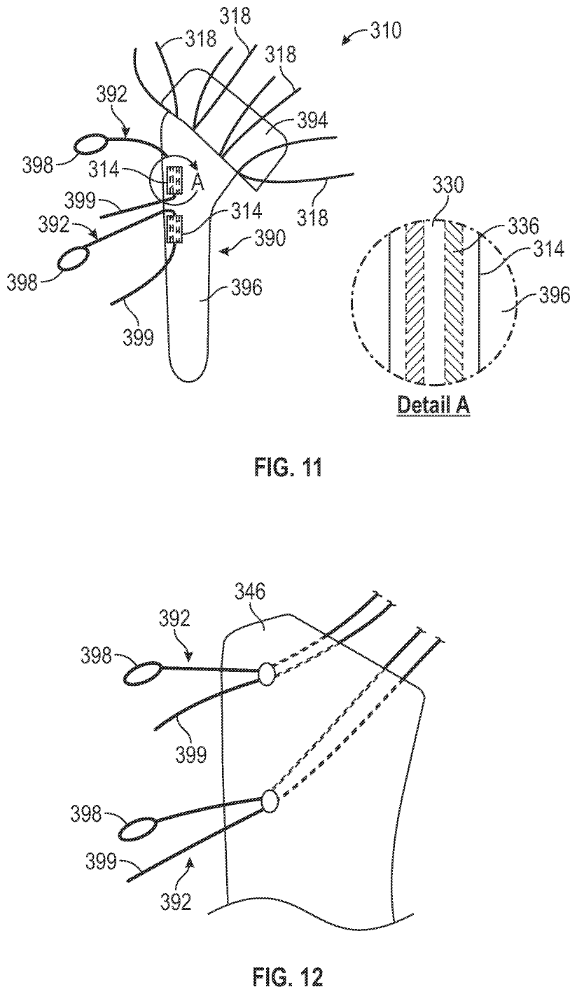

FIG. 11 illustrates another exemplary suture locking system 310 that can be used when performing various tensionable knotless surgical procedures (e.g., shoulder arthroplasty procedures). The suture locking system 310 may include an arthroplasty implant 390, one or more locking ferrules 314 integrated with or attached to the arthroplasty implant 390, a shuttle device 392 passed through each locking ferrule 314, and one or more sutures 318 attached to the arthroplasty implant 390.

The arthroplasty implant 390 may include a cup portion 394 and a stem portion 396 that extends from the cup portion 394. The size, shape, and material-makeup of the arthroplasty implant 390 are not intended to limit this disclosure.

Each locking ferrule 314 may be connected to the stem portion 396 of the arthroplasty implant 390. In an embodiment, the locking ferrules 314 are formed as integral features of the stem portion 396. In another embodiment, the locking ferrules 314 are secured within pockets formed in the stem portion 396. The locking ferrules 314 may be connected to the stem portion 396 in any manner.

Each locking ferrule 314 may include a cannulation 330 that includes a plurality of locking barbs 336 (see inset A of FIG. 11). The locking barbs 336 may be arranged to establish a one-way locking mechanism for locking one or more of the sutures 318 relative to the arthroplasty implant 390 during a surgical procedure.

Each shuttle device 392 may be a passing wire or another suture, for example. Each shuttle device 392 may be pre-connected (e.g., by positioning the shuttle device 392 through the cannulation 330) to one of the locking ferrules 314 and may include an eyelet 398 and a free end 399.

Each suture 318 may be preconnected to the arthroplasty implant 390. In an embodiment, the sutures 318 are connected to the cup portion 394 of the arthroplasty implant 390. For example, the sutures 318 may be passed through one or more suture openings formed through the cup portion 394.

FIGS. 12, 13, and 14 schematically illustrate an exemplary surgical method during which the suture locking system 310 of FIG. 11 may be utilized to tension and lock the sutures 318 as part of an arthroplasty procedure. In an embodiment, the surgical method may involve securing a tissue 346 (e.g., a subscapularis muscle) to a bone 348 (e.g., a humerus) as part of the arthroplasty procedure.

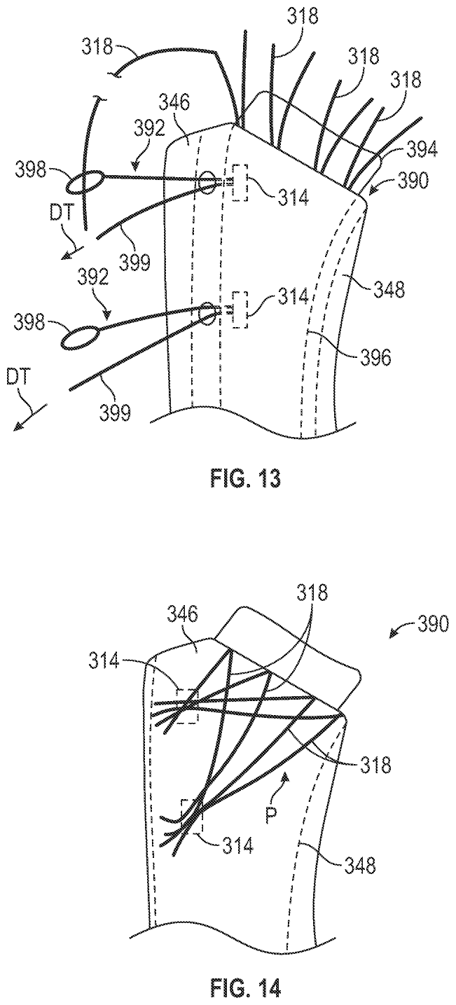

Referring first to FIG. 12, the surgical method may include passing each shuttle device 392 (which is preconnected to one of the locking ferrules 314) through the tissue 346. Each shuttle device 392 may be positioned such that both the eyelet 398 and the free end 399 are accessible from a location outside of the tissue 346. The stem portion 396 of the arthroplasty implant 390 may then be inserted into the medullary canal of the bone 348 as is schematically shown in FIG. 13.

Referring next to FIGS. 12 and 13, the sutures 318 may be passed through the eyelet 398 of one of the shuttle devices 392, and the free end 399 of the shuttle device 392 may then be pulled (in a direction DT) to shuttle each suture 318 through one of the locking ferrules 314. The sutures 318 may then be tensioned to knotlessly lock each suture 318 relative to its respective locking ferrule 314 and thereby reduce and secure the tissue 346 at a desired position relative to the bone 348.

The sutures 318 may be arranged in various bridging configurations over top of the tissue 346 via the locking ferrules 314. For example, the sutures 318 may be arranged in a crisscross pattern P that provides a desired area of footprint compression over top of the tissue 346.

The suture locking systems of this disclosure may be utilized for performing various tensionable knotless surgical repairs. The suture locking systems provide for tensioning and retensioning suture(s) at various points of the repair, including subsequent to implantation of accompanying fixation devices, thus providing numerous advantages over prior tissue repair systems and techniques.

Although the different non-limiting embodiments are illustrated as having specific components or steps, the embodiments of this disclosure are not limited to those particular combinations. It is possible to use some of the components or features from any of the non-limiting embodiments in combination with features or components from any of the other non-limiting embodiments.

It should be understood that like reference numerals identify corresponding or similar elements throughout the several drawings. It should further be understood that although a particular component arrangement is disclosed and illustrated in these exemplary embodiments, other arrangements could also benefit from the teachings of this disclosure.

The foregoing description shall be interpreted as illustrative and not in any limiting sense. A worker of ordinary skill in the art would understand that certain modifications could come within the scope of this disclosure. For these reasons, the following claims should be studied to determine the true scope and content of this disclosure.

What is claimed is:

1. A suture locking system, comprising:
a surgical button;
a locking ferrule that includes a one-way locking mechanism;
a flexible coupling that extends between the surgical button and the locking ferrule; and
a suture received through the locking ferrule and locked relative to the locking ferrule by the one-way locking mechanism.

2. The suture locking system as recited in claim 1, wherein the one-way locking mechanism is configured to allow the suture to be tensioned in a first direction and prevent the suture from moving in a second direction.

3. The suture locking system as recited in claim 2, wherein the one-way locking mechanism is established by a plurality of locking barbs provided within a cannulation of the locking ferrule.

4. The suture locking system as recited in claim 1, wherein the flexible coupling is adjustable to reduce a distance between the surgical button and the locking ferrule.

5. The suture locking system as recited in claim 4, wherein the flexible coupling is a second suture that is separate and distinct from the suture.

6. The suture locking system as recited in claim 4, wherein the flexible coupling is an adjustable suture loop.

7. A suture locking system, comprising:
a bone plate including an opening;
a locking ferrule received within the opening and including a one-way locking mechanism; and
a suture received through the locking ferrule and locked relative to the bone plate by the one-way locking mechanism.

8. The suture locking system as recited in claim 7, wherein the one-way locking mechanism is configured to allow the suture to be tensioned in a first direction and prevent the suture from moving in a second direction.

9. The suture locking system as recited in claim 8, wherein the one-way locking mechanism is established by a plurality of locking barbs provided within a cannulation of the locking ferrule.

10. The suture locking system as recited in claim 9, wherein the cannulation extends through an eyelet portion of the locking ferrule.

11. The suture locking system as recited in claim 10, comprising an engagement portion connected to the eyelet portion.

12. The suture locking system as recited in claim 11, wherein the engagement portion includes a first thread that is configured to engage a second thread of the opening of the bone plate.

13. The suture locking system as recited in claim 7, wherein the locking ferrule includes an engagement portion received within the opening and an eyelet portion that extends to a location outside of the opening.

14. The suture locking system as recited in claim 13, wherein the eyelet portion provides the one-way locking mechanism.

15. The suture locking system as recited in claim 14, wherein the one-way locking mechanism includes a plurality of locking barbs that allow the suture to be tensioned in a first direction and prevent the suture from moving in a second direction.

16. A suture locking system, comprising:
an arthroplasty implant;
a locking ferrule connected to the arthroplasty implant and including a one-way locking mechanism; and
a shuttle device passed through the locking ferrule.

17. The suture locking system as recited in claim 16, wherein the shuttle device is configured to shuttle a suture through the locking ferrule.

18. The suture locking system as recited in claim 17, wherein the shuttle device includes an eyelet and a free end.

19. The suture locking system as recited in claim 17, wherein the one-way locking mechanism is configured to allow the suture to be tensioned in a first direction and prevent the suture from moving in a second direction.

20. The suture locking system as recited in claim 19, wherein the one-way locking mechanism is established by a plurality of locking barbs provided within a cannulation of the locking ferrule.
